(19) United States
(12) Patent Application Publication
(10) Pub. No.: US 2010/0215624 A1
(43) Pub. Date: Aug. 26, 2010

(54) Pharmaceutical Compositions for Treating Rheumatoid Arthritis Comprising Semi-Mature Dendritic Cell

(75) Inventors: Mi-Sun Kang, Seoul (KR); Dae-Seog Lim, Gyeonggi-do (KR); Ju-Ah Jeong, Seoul (KR); Hyun-Soo Lee, Seoul (KR); Yong-Soo Bae, Gyeonggi-do (KR)

Correspondence Address: CLARK & ELBING LLP, 101 FEDERAL STREET, BOSTON, MA, 02110, US

(73) Assignee: CREAGENE INC., Sungnam-si (KR)

(21) Appl. No.: 12/597,259

(22) PCT Filed: Mar. 12, 2008
(86) PCT No.: PCT/KR08/01397

§ 371 Date: Dec. 21, 2009

(30) Foreign Application Priority Data
Apr. 24, 2007 (KR) 10-2007-0039629

(51) Int. Cl. A61K 35/30; C12N 5/0784; A61P 29/00; A61P 19/00

(52) U.S. Cl. 424/93.7; 435/354

(57) ABSTRACT

The present invention relates to a pharmaceutical composition for treating rheumatoid arthritis, which comprises (a) a pharmaceutically effective amount of a semi-mature dendritic cell; and (b) a pharmaceutically acceptable carrier. The semi-mature dendritic cell of this invention has a safe and remarkably improved potential to treat or prevent rheumatoid arthritis through the activity of the suppression of auto-immune responses.

### BACKGROUND OF THE INVENTION

1. Field of the Invention

The present invention relates to a pharmaceutical composition for treating or preventing autoimmune disease, in particular rheumatoid arthritis comprising a semi-mature dendritic cell as an active ingredient.

2. Description of the Related Art

Dendritic cells (DCs) are known as professional antigen presenting cells. DCs are responsible for some of the initial pathogenic recognition process, sampling the environment and differentiating depending on the concentration of signals. The DCs' function is to collect antigen from pathogens and host cells in tissues, and to present multiple antigen samples to naive T-cells in the lymph node.

DCs exist in a number of different states of maturity, dependent on the type of environmental signals present in the surrounding fluid. They can exist in either immature, semi-mature or mature forms.

Immature DCs (imDCs) are cells found in their initial maturation state. They reside in the tissue where their primary function is to collect and remove debris from the interstitial fluid. The ingested material is then processed by the cell. It is either metabolised for use by the cell, returned to the environment, or is repackaged for presentation to another immune cell. At this point the matter can be termed antigen, and could be a ‘self’ molecule or something foreign. The representation of antigenic material is performed by complexing the antigen with another molecule namely the MHC molecule family, necessary for binding to T cell receptors. In order to present antigen to T-cells, DC needs sufficient antigen presented with MHC. However, the expression of inflammatory cytokines is needed in order to activate T-cells. Therefore, a T-cell encounter with an imDC results in the deactivation of the T-cell. Differentiation of imDCs occurs in response to the receipt of various signals. This leads to full or partial maturation depending on the combination of signals received.

Due to the low levels of inflammatory cytokines expressed by imDCs, they are not able to activate T-cells on contact. In order to present antigen and activate T cells, the increased expression (or up-regulation) of a number of proteins and cytokines is necessary. DCs which have the ability to activate naive T-cells are termed mature DCs (mDCs). For imDCs to differentiate and become mDCs, the imDCs have to be exposed to a certain number of signals. This includes activation of toll-like receptors through exposure to both the exogenous and endogenous signals. On exposure to various combinations of these signals, the DC up-regulates a number of molecules vital for stimulating a T-cell response. Perhaps most importantly, it up-regulates a number of costimulatory molecules, pro-inflammatory cytokines (namely IL-12), and migrates from the tissue to the local draining lymph node. During this migration period, the imDC changes morphologically too. Instead of being compact, the DC develops whispy, finger-like projections-characterising it as mDCs (see FIG. 7) (Greensmith, Julie and Aickelin, Uwe and Cayzer, Steve (2005) ‘Introducing Dendritic Cells as a Novel Immune-Inspired Algorithm for Anomaly Detection’. In: ICARIS-2005, 4th International Conference on Artificial Immune Systems, LNCS 3627, 2005, Banff, Canada).

During the antigen collection process, imDCs can experience other environmental conditions. This can affect the end-stage differentiation of a DC. These different conditions can give rise to semi-mature DCs (smDCs). The signals responsible for producing smDCs are also generated by the tissue-endogenous signals. During the process of apoptosis, a number of proteins are actively up-regulated and secreted by the dying cell. The release of TNF (tumor necrosis factor)-α from apoptosing cells is thought to be one candidate responsible for creating semimature DCs (Manfred B. Lutz and Gerold Schuler. Immature, semi-mature and fully mature dendritic cells: which signals induce tolerance or immunity? Trends in Immunology, 23(9):991-1045, 2002).

As a result of exposure to apoptotic cytokines including TNF-α, imDCs also undergo migration to the lymph node, and some maturation as shown in FIG. 7. Costimulatory molecules are up-regulated by a small yet significant amount and, after migration to the lymph node, the cell can present antigen to any matching T-cell. However, smDCs do not produce any great amount of pro-inflammatory cytokines, necessary for promoting activation of T cells. Instead, smDCs can produce small quantities of IL-10 (anti-inflammatory cytokine), which acts to suppress matching T-cells.

Autoimmunity results from a breakdown in the regulation in the immune system resulting in an inflammatory response directed at self-antigens and tissues. The autoimmune diseases involving the destruction of self-antigen by T lymphocytes include the multiple sclerosis, insulin-dependent diabetes mellitus (also referred to as ‘IDDM’ or ‘type I DM’) and the rheumatoid arthritis, etc (K J Johnson et al., Immunopathology in Pathology, pp. 104-153 (1999)).

For example, in the rheumatoid arthritis represented by a systemic chronic autoimmune disease, inflammation in joint continually infiltrates into cartilage and osteoid tissue resulting in bone corrosion. Type II collagen, a major constituent of joint, is well-known antigen causing arthritis and there is a publication showing that type II collagen causes rheumatoid arthritis in mice having specific MHC antigen (L K Myers et al., Life Sci., 19: 1861-1878 (1997)). In rheumatoid patient, the amount of cytokines secreted from macrophage or fibroblast is increased, and Th1 specific cytokines including IFN-γ and IL-2 are also accentuated. The Th1 specific cytokines are known to exacerbate arthritis contrary to the arthritis-prophylactic effect of Th2 cytokines comprising IL-4 and IL-10. Furthermore, S H Kim et al. showed that injecting into leg of artificially arthritis-induced mouse viral vectors expressing Th2 cytokines, IL-4 or IL-10, provided treatment effect for arthritis even in non-injected leg as well as injected one (S H Kim, et al., J. Immunol, 166: 3499-3505 (2001)).

Currently, the drugs for treating or alleviating rheumatoid arthritis include methotrexate, azathioprine, cyclophosphamide and corticosteroid (Johnson C J et al., Ann. Pharmacother., 35 (4): 464-471 (2001); and Seymour H E et al., Br. J. Clin. Pharmacol., 51 (3): 201-208 (2001)). However, the described drugs are incapable of preventing the destruction of the joint efficiently and have several side effects as well.

Meanwhile, M. B. Lutz et al. have reported that injections of DCs matured with tumor necrosis factor-α induce antigen-specific protection from experimental autoimmune encephalomyelitis (EAE) in C57BL/6 mice. However, they did not disclose any possibility of DCs to treat rheumatoid arthritis (Menges, M., S. Rossner, C. Voigtlander, H. Schindler, N. A. Kukutsch, C. Bogdan, K. Erb, G. Schuler, and M. B. Lutz: Repetitive injections of dendritic cells matured with tumor necrosis factor-α induce antigen-specific protection of mice from autoimmunity. J. Exp. Med, 2002, 195: 15-21).

Throughout this application, various patents and publications are referenced and citations are provided in parentheses. The disclosure of these patents and publications in their entities are hereby incorporated by references into this application in order to more fully describe this invention and the state of the art to which this invention pertains.

### DETAILED DESCRIPTION OF THIS INVENTION

Based on the facts that antigen specific semi-mature dendritic cells (smDCs) can induce immune-tolerance responses, the present inventors have prepared smDCs by incompletely maturing immature dendritic cells derived from mouse bone marrow via culturing it in the presence of proper cytokines and antigens, and have demonstrated that the obtained smDCs induce immune-tolerance responses and exhibit a remarkable potential to treat rheumatoid arthritis.

Accordingly, it is an object of this invention to provide a semi-mature dendritic cell for treating or preventing rheumatoid arthritis.

It is another object of this invention to provide a pharmaceutical composition comprising a semi-mature dendritic cell for treating or preventing rheumatoid arthritis.

Other objects and advantages of the present invention will become apparent from the following detailed description together with the appended claims and drawings.

In one aspect of this invention, there is provided a semi-mature dendritic cell for treating rheumatoid arthritis.

In another aspect of this invention, there is provided a pharmaceutical composition for treating rheumatoid arthritis, which comprises (a) a pharmaceutically effective amount of a semi-mature dendritic cell; and (b) a pharmaceutically acceptable carrier.

Based on the facts that antigen specific semi-mature dendritic cells (smDCs) can induce immune-tolerance responses, the present inventors have prepared smDCs by incompletely maturing immature dendritic cells derived from mouse bone marrow via culturing it in the presence of proper cytokines and antigens, and have demonstrated that the obtained smDCs induce immune-tolerance responses and exhibit a remarkable potential to treat rheumatoid arthritis.

As used herein, the term “dendritic cells (DCs)” refers to professional antigen-presenting cells that can internalize antigen and process the antigen, such that the antigen, or peptide derived from the antigen, is presented in the context of both MHC (major histocompatibility complex) class I complex and the MHC class II complexes. Dendritic cells of this invention typically has the phenotype and characteristics of the DCs described in Steinman et al., Annual Rev. Immunol. 9:271-296, 1991 and in Banchereau and Steinman Nature 392:245-252, 1998. Dendritic cells include both immunogenic and tolerogenic antigen presenting cells, and may be classified as immature, semi-mature, or fully mature.

As used herein, the term “immature dendritic cells (imDCs)” refers to dendritic cells that lack the cell surface markers found on mature DCs, such as CD83 and CD14; express low levels of CCR7 and the cytosolic protein DC-LAMP, and low levels of the costimulatory molecules CD40, CD80 and CD 86, and usually express CD1a and CCR1, CCR2, CCR5 and CXCR1.

As used herein, the term “mature dendritic cells (mDCs)” refers to a population of dendritic cells which are matured from imDCs and have increased expression of MHC class 11, CD40, CD80, CD83 and CD86 as well as DC-LAMP; are characterized by their release of proinflammatory cytokines, and their ability to cause increased proliferation of naive allogeneic T cells and/or increased production of DC cytokines in a mixed lymphocyte reaction. Mature DCs typically express high levels of CCR7, and CXCR4 and low levels of CCR1 and CCR5.

As used herein, the term “semi-mature dendritic cells (smDCs)” refers to DCs that are partially or incompletely matured from immature dendritic cells so that they have lost some of the characteristics of immature DCs but do not have all the characteristics of a mature DC phenotype and are characterized by their ability to induce a tolerogenic immune response to self-antigens.

The expression profiling of surface markers of DCs is able to be carried out by the flow cytometry analysis known to those skilled in the art.

The semi-mature dendritic cells contained in the pharmaceutical composition of this invention can be obtained by inducing the differentiation of immature DCs.

General procedures for isolating and culturing immature DCs are disclosed in U.S. Pat. No. 5,994,126 and WO 97/29182, which are incorporated herein by references.

Suitable source for isolating immature dendritic cells is tissue that contains immature dendritic cells or their progenitors, and specifically include spleen, afferent lymph, bone marrow, blood, and cord blood, as well as blood cells elicited after administration of cytokines such as G-CSF or FLT-3 ligand.

According to a specific embodiment of this invention, a tissue source may be treated prior to culturing with substances that stimulate hematopoiesis, such as, for example, G-CSF, FLT-3, GM-CSF, M-CSF, TGF-β, and thrombopoietin in order to increase the proportion of dendritic cell precursors relative to other cell types.

Such pretreatment may also remove cells which may compete with the proliferation of the dendritic cell precursors or inhibit their survival. Pretreatment may also be used to make the tissue source more suitable for in vitro culture. Those skilled in the art would recognize that the method of treatment will likely depend on the particular tissue source. For example, spleen or bone marrow would first be treated so as to obtain single cells followed by suitable cell separation techniques to separate leukocytes from other cell types as described in U.S. Pat. Nos. 5,851,756 and 5,994,126 which are herein incorporated by references. Treatment of blood would preferably involve cell separation techniques to separate leukocytes from other cell types including red blood cells (RBCs) which are toxic. Removal of RBCs may be accomplished by standard methods known in the art. According to a preferred embodiment of the invention, the tissue source is blood or bone marrow.

According to a further embodiment, immature dendritic cells are derived from multipotent blood monocyte precursors (see WO 97/29182). These multipotent cells typically express CD14, CD32, CD68 and CD115 monocyte markers with little or no expression of CD83, or p55 or accessory molecules such as CD40 and CD86. When cultured in the presence of cytokines such as a combination of GM-CSF and IL-4 or IL-13 as described below, the multipotent cells give rise to the immature dendritic cells. The immature dendritic cells can be modified, for example using vectors expressing IL-10 to keep them in an immature state in vitro or in vivo. Those skilled in the art would recognize that any number of modifications may be introduced to the disclosed methods for isolating immature dendritic cells and maintaining them in an immature state in vitro and in vivo having regard to the objects of the several embodiments of the invention here disclosed.

Cells obtained from the appropriate tissue source are cultured to form a primary culture, preferably, on an appropriate substrate in a culture medium supplemented with granulocyte/macrophage colony-stimulating factor (GM-CSF), a substance which promotes the differentiation of pluripotent cells to immature dendritic cells as described in U.S. Pat. Nos. 5,851,756 and 5,994,126 which are herein incorporated by references. In a preferred embodiment, the substrate would include any tissue compatible surface to which cells may adhere. Preferably, the substrate is commercial plastic treated for use in tissue culture.

To further increase the yield of immature dendritic cells, other factors, in addition to GM-CSF, may be added to the culture medium which block or inhibit proliferation of non-dendritic cell types. Examples of factors which inhibit non-dendritic cell proliferation include interleukin-4 (IL-4) and/or interleukin-13 (IL-13), which are known to inhibit macrophage proliferation. The combination of these substances increases the number of immature dendritic cells present in the culture by preferentially stimulating proliferation of the dendritic cell precursors, while at the same time inhibiting growth of non-dendritic cell types.

According to a specific example of the invention, an enriched population of immature dendritic cells can be generated from blood monocyte precursors by plating mononuclear cells on plastic tissue culture plates and allowing them to adhere. The plastic adherent cells are then cultured in the presence of GM-CSF and IL-4 in order to expand the population of immature dendritic cells. Other cytokines such as IL-13 may be employed instead of using IL-4. In order to increase the population of immature DCs, plastic adherent cells are plated with low density (2×10⁵ cells/ml) in the presence of GM-CSF and cultured for 10 days. If cells are cultured for 7 days, the yield of differentiation of DCs from mononuclear progenitor cells is 70%. However, the above method can significantly increase the yield of the differentiation of imDCs or mDCs from mononuclear progenitor cells up to 90-95%.

A medium useful in the procedure of obtaining immature dendritic cells includes any conventional medium for culturing animal cells, preferably, a medium containing serum (e.g., fetal bovine serum, horse serum and human serum). The medium used in this invention includes, for example, RPMI series (e.g., RPMI 1640), Eagles's MEM (Eagle's minimum essential medium, Eagle, H. Science 130:432 (1959)), α-MEM (Stanner, C. P. et al., Nat. New Biol. 230:52 (1971)), Iscove's MEM (Iscove, N. et al., J. Exp. Med. 147:923 (1978)), 199 medium (Morgan et al., Proc. Soc. Exp. Bio. Med. 73:1 (1950)), CMRL 1066, RPMI 1640 (Moore et al., J. Amer. Med. Assoc. 199:519 (1967)), F12 (Ham, Proc. Natl. Acad. Sci. USA 53:288 (1965)), F10 (Ham, R. G. Exp. Cell Res. 29:515 (1963)), DMEM (Dulbecco's modification of Eagle's medium, Dulbecco, R. et al., Virology 8:396 (1959)), Mixture of DMEM and F12 (Barnes, D. et al., Anal. Biochem. 102:255 (1980)), Way-mouth's MB752/1 (Waymouth, C. J. Natl. Cancer Inst. 22:1003 (1959)), McCoy's 5A (McCoy, T. A., et al., Proc. Soc. Exp. Biol. Med. 100:115 (1959)) and MCDB series (Ham, R. G. et al., In Vitro 14:11 (1978)) but not limited to. The medium may contain other components, for example, antioxidant (e.g., β-mercaptoethanol). The detailed description of media is found in R. Ian Freshney, Culture of Animal Cells, A Manual of Basic Technique, Alan R. Liss, Inc., New York, the teaching of which is incorporated herein by reference in its entity.

The immature DCs that used in this invention may be obtained from organ, tissue, bone-marrow, or blood of animal. In addition, the immature DCs used in the invention may be syngenic or allogenic.

According to a preferred embodiment of this invention, semi-mature DCs included in the present composition may be obtained by culturing immature DCs in the presence of suitable cytokines. Preferably, the suitable cytokine is TNF-α.

In general, for dendritic cells generated from any precursor, when incubated in the presence of activation factors such as monocyte-derived cytokines, lipopolysaccharide and DNA containing CpG repeats, cytokines such as TNF-α, IL-6, IFN-α, IL-1β, necrotic cells, readherence, whole bacteria, membrane components, RNA or polyI:C (polyinosinic-polycytidylic acid), immature dendritic cells will become activated (Clark, R. B. (2002). J Leukoc Biol, 71, 388-400; Hacker, G., Redecke, V. & Hacker, H. (2002). Immunology 105, 245-251; Kaisho, T. & Akira, S. (2002). Biochim Biophys Acta 1589, 1-13; Koski, G. K., Lyakh, L. A., Cohen, P. A. & Rice, N. R. (2001). Crit Rev Immuno/21, 179-189). This process of dendritic cell activation is inhibited in the presence of NF-κB inhibitors (O'Sullivan, B. J., and Thomas, R (2002). CD40 Ligation conditions dendritic cell antigen-presenting function through sustained activation of NF-kappaB, J Immunol 168, 5491-5498.).

The semi-mature DCs of the present pharmaceutical composition are useful for inducing self-antigen specific tolerogenic immune response. The exemplary self-antigen which may be used in the practice of the present invention include, but not limited to, self antigens that are target of autoimmune responses, allergens and transplantation antigens.

Examples of self antigens include, but are not restricted to, lupus autoantigen, Smith, Ro, La, U1-RNP, fibrillin (scleroderma), GAD65 (diabetes related), insulin, myelin basic protein, histones, PLP, collagen, glucose-6-phosphate isomerase, citrullinated proteins and peptides, thyroglobulin, various tRNA synthetases, acetyl choline receptor (AchR), MOG, proteinase-3, myeloperoxidase etc.

Examples of allergens include, but are not limited to, Fel d 1 (i.e., the feline skin and salivary gland allergen of the domestic cat Felis domesticus, the amino acid sequence of which is disclosed International Publication WO 91/06571), Der p I, Der p II, Der fI or Der fII (i.e., the major protein allergens from the house dust mite dermatophagoides, the amino acid sequence of which is, disclosed in International Publication WO 94/24281). Other allergens may be derived, for example from the following: grass, tree and weed (including ragweed) pollens; fungi and moulds; foods such as fish, shellfish, crab, lobster, peanuts, nuts, wheat gluten, eggs and milk; stinging insects such as bee, wasp, and hornet and the chirnomidae (non-biting midges); other insects such as the housefly, fruitfly, sheep blow fly, screw worm fly, grain weevil, silkworm, honeybee, non-biting midge larvae, bee moth larvae, mealworm, cock-roach and larvae of Tenibrio molitor, beetle; spiders and mites, including the house dust mite; allergens found in the dander, urine, saliva, blood or other bodily fluid of mammals such as cat, dog, cow, pig, sheep, horse, rabbit, rat, guinea pig, mouse and gerbil; airborne particulates in general; latex; and protein detergent additives.

Transplantation antigens can be derived from donor cells or tissues or from the donor antigen-presenting cells bearing MHC loaded with self antigen in the absence of exogenous antigen.

Antigen-specific semi-mature dendritic cell can be produced by contacting a immature dendritic cell with at least one antigen that corresponds to a specified target antigen, or with a polynucleotide from which the antigen is expressible, for a time and under conditions sufficient for the antigen or a processed form thereof to be presented by the dendritic cell. Preferably, the contact can be carried out by co-culture.

According to a preferred embodiment of this invention, the semi-mature DCs can be obtained by co-culturing immature DCs with TNF-α, together with antigen. Preferably, the concentration of TNF-α is 10-1000 U/ml, more preferably 50-800 U/ml, still more preferably 200-700 U/ml, most preferably 500 U/ml. The co-culture time is preferably 1-10 hr, more preferably 2-8 hr, still more preferably 3-7 hr, most preferably 4 hr.

Preferably, the self-antigen is rheumatoid arthritis specific self-antigen, more preferably collagen.

The culture media, which can be used to prepare semi-mature dendritic cells by co-culturing with the specific antigen, is the same one that is used for preparing immature dendritic cells.

According to a preferred embodiment of this invention, the semi-mature dendritic cells of the invention have reduced expression level of one or more of co-stimulating factors of CD 80, CD86 and CD40 compared to mature dendritic cells.

The semi-mature dendritic cells can be identified based on typical morphologies and low expression level of co-stimulating factors of CD 80, CD86 and CD40 compared to that of mature dendritic cells. Thus, by utilizing standard antibody staining techniques known in the art, it is possible to assess the proportion of semi-mature dendritic cells in any given culture. Antibodies may also be used to isolate or purify semi-mature dendritic cells from mixed cell cultures by flow cytometry or other cell sorting techniques well known in the art.

In this invention, the autoimmune diseases therapeutically applicable by the semi-mature DCs include any disease or disorder caused by autoimmune response comprising type I diabetes mellitus, rheumatoid arthritis, multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, diopathic thrombocytopenia purpura, cirrhosis, pemphigus vulgaris, autoimmune infertility, Goodpasture's disease, bullous pemphigoid, discoid lupus, ulcerative colitis and dense deposit disease. Preferably, the applicable disease or disorder of the pharmaceutical composition of this invention is rheumatoid arthritis.

The semi-mature DCs of this invention exert significantly enhanced activity to suppress immune responses. The immune tolerance induced by the semi-mature DCs of this invention is the result of immunosuppressive effect exerted by CD4⁺ CD25⁺ Foxp3⁺ specific T_{reg} cells. T_{reg} cells have been reported to suppress the activities, proliferation, differentiation and effector function of the various types of immune cell including CD4⁺ and CD8⁺ T cells, B cells, NK cells and dendritic cells (Sakaguchi, S. 2005. Naturally arising Foxp3-expressing CD25⁺ CD4⁺ regulatory T cells in immunological tolerance to self and non-self. Nat. Immunol. 6:345-352). Although the mechanism of immune suppression induced by T_{reg} cell has not been exactly elucidated, it is well known that T_{reg} cell exerts its immunosuppressive effect through the induction of immunosuppressive cytokines such as TGF-β and IL-10, or the cell to cell interactions mediated by suppressive receptor CTLA-4 (Ghiringhelli, F., C. Menard, M. Terme, C. Flament, J. Taieb, N. Chaput, P. E. Puig, S. Novault, B. Escudier, E. Vivier, et al. 2005. CD4+ CD25+ regulatory T cells inhibit natural killer cell functions in a transforming growth factor-beta-dependent manner. J. Exp. Med. 202:1075-1085; Meirelles Lda, S., and N. B. Nardi. 2003. Murine marrow-derived mesenchymal stem cell: isolation, in vitro expansion, and characterization. Br J Haemato/123:702-711).

The semi-mature dendritic cells of the instant invention significantly increase a population of CD25⁺ Foxp3⁺ T_{reg} cell which exhibit an immunosuppressive activity and remarkably enhance the secretion of an immunosuppressive cytokine TGF-β. In addition, the dendritic cells of this invention suppress the secretion of IFN-γ (Th1 cytokine) and promote the secretion of IL-4 and IL-10 (Th2 cytokine), and as a result decrease the ratio of Th1/Th2. As a result, semi-mature dendritic cells of this invention can induce in vivo immunosuppressive response.

According to a preferred embodiment of this invention, semi-mature DCs in the present pharmaceutical composition have a potential to increase the population of CD25⁺ Foxp3⁺ T_{reg} cell.

According to another preferred embodiment of this invention, semi-mature DCs in the present pharmaceutical composition have a potential to suppress the secretion of IFN-γ and to promote the secretion of IL-4 and IL-10.

According to another preferred embodiment of this invention, semi-mature DCs in the present pharmaceutical composition have a potential to increase the secretion of an immunosuppressive cytokine TGF-β.

The term used herein “pharmaceutically effective amount” means a sufficient amount to exert pharmaceutical effects. According to a preferred embodiment of this invention, the number of semi-mature DCs in the composition is, but not limited to, preferably less than 2×10⁶ Cells, more preferably less than 1×10⁶ Cells, still more preferably less than 5×10⁵ Cells, even more preferably 5×10⁵-2×10⁵ Cells, most preferably 2×10⁵ Cells.

In the pharmaceutical compositions of this invention, the pharmaceutically acceptable carrier may be conventional one for formulation, including lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methyl hydroxy benzoate, propyl hydroxy benzoate, talc, magnesium stearate, and mineral oils, but not limited to. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

A suitable dose of the pharmaceutical composition of the present invention may vary depending on pharmaceutical formulation methods, administration methods, the patient's age, body weight, sex, severity of diseases, diet, administration time, administration route, an excretion rate and sensitivity for a used pharmaceutical composition. Preferably, the pharmaceutical composition of the present invention is administered with a daily dose of 0.001-100 mg/kg (body weight).

According to the conventional techniques known to those skilled in the art, the pharmaceutical composition may be formulated with pharmaceutically acceptable carrier and/or vehicle as described above, finally providing several forms including a unit dose form and a multi-dose form.

The pharmaceutical composition according to the present invention may be administered via the oral or parenterally. When the pharmaceutical composition of the present invention is administered parenterally, it can be done by intravenous, intraperitoneal, intramuscular, subcutaneous, or local administration. It is desirable that the route of administration of the present composition should be determined according to the disease to which the composition of this invention is applied. For example, where the present composition is used to treat or prevent patients suffering from rheumatoid arthritis, it is preferably administered via the intravenous, most preferably injected into the joint via local administration.

The features and advantages of this invention can be summarized as follows:

(i) The present invention provides a pharmaceutical composition for treating or preventing rheumatoid arthritis comprising semi-mature dendritic cells having a remarkable activity to induce antigen specific tolerogenic immune responses.

(ii) The instant pharmaceutical composition having an activity to suppress autoimmune responses can be utilized for treating or preventing autoimmune diseases, in particular rheumatoid arthritis.

### BRIEF DESCRIPTION OF THE DRAWINGS

FIG. 1 shows that DCs stimulated with TNF-α represent phenotypes of semi-mature DCs.

FIG. 2a-2b show the immunosuppressive characteristics of semi-mature DCs.

FIG. 3 shows results that semi-mature DCs induce inhibition of CIA developments.

FIG. 4 shows results of histological analysis of the mice hind paws.

FIG. 5 shows that semi-mature DCs suppress CIA at least through the generation of T_{reg} cells and augmentation of the Th2 response.

FIG. 6 represents that the increase of T_{reg} cell population and the low expression of costimulatory molecules are dependent on the gradual decrease of semi-mature DC numbers.

FIG. 7 shows the Environmental Scanning Electron Microscope (ESEM) images of immature, semi-mature and mature DCs respectively.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Materials and Methods

### Mice and Reagents

Pathogen-free female DBA/1 mice, 6 to 7 weeks of age, were purchased from Orient Bio (Gyeonggi, Republic of Korea) and maintained in the Animal Maintenance Facility of the CreaGene Research Institute (Gyeonggi, Republic of Korea). Five or six mice were housed per cage under standard conditions of temperature and light, and were fed standard laboratory chow and water ad libitum. Recombinant mouse (rm) TNF-α was purchased from R&D systems (Abington, OX, UK) and rmGM-CSF was purchased from CreaGene (Gyeonggi, Republic of Korea). CFA (complete Freund's adjuvant), IFA (incomplete Freund's adjuvant), collagen (type II collagen, chicken), and LPS (lipopolysaccharide) were purchased from Sigma-Aldrich (St Louis, Mo., USA).

The culture medium used was RPMI 1640 (GIBCO Laboratories, Grand Island, N.Y., USA) supplemented with 10% FBS (GIBCO Laboratories, Grand Island, N.Y., USA), 50 μM 2-mercaptoethanol (Life technologies, Gaithersburg, Md., USA), 50 μg/ml streptomycin, 50 U/ml penicillin, and 25 μg/ml amphotericin B (GIBCO Laboratories; Grand Island, N.Y., USA). For immunofluorescence staining, PE-conjugated anti-mouse CD11c and CD25, and FITC-conjugated anti-mouse MHC class II, CD80, CD86, CD40, CD54, CD14, and CD3 were purchased from BD Pharmingen (San Diego, Calif., USA). FITC-conjugated anti-mouse Foxp3 was purchased from eBioscience (San Diego, Calif., USA).

### DC Generation

DCs were generated from bone marrow (BM) progenitors of DBA/1 mice as descried by Lutz et al. [Lutz, M. B., N, Kukutsch, A. L. Ogilvie, S. Rossner, F. Koch, N. Romani, and G. Schuler: An advanced culture method for generating large quantities of highly pure dendritic cells from mouse bone marrow. J. Immunol. Methods, 1999, 223:77-92.]. Briefly, BM single cell suspensions were prepared and the washed cells were cultured by plating on the tissue culture plate with the concentration of 2×10⁵ cells/ml in 10 ml of media supplemented with 20 ng/ml GM-CSF. Cells were fed with 10 ml of fresh medium and 20 ng/ml of GM-CSF at 3 days. Cells were further fed with 10 ml of fresh medium and 20 ng/ml of GM-CSF at days of 6 and 8. After 10 days of culture, smDCs were generated by additional culturing for 4 hr in fresh media in the presence of 10 ng/ml GM-CSF, 500 U/ml TNF-α and 50 μg/ml collagen, and mature DCs were also generated by further culturing for 24 hr with the addition of 1 μg/ml LPS and 50 μg/ml collagen.

### Mixed Lymphocyte Reaction (MLR)

Splenocytes were isolated from the spleen of DBA/1 mice and disaggregated into RPMI 1640 medium. Additionally, CD4⁺ T cells were isolated from splenocytes by use of a CD4 MicroBeads mouse kit (Miltenyi Biotec, Auburn, Calif., USA). Briefly, CD4⁺ T cells were separated by passing the cell suspension over a magnetic-activated cell sorter MS column held in MACS magnetic separator (Miltenyi Biotec, Auburn, Calif., USA). The CD4⁺ T cells adhering to the column were then used for this assay. Splenocytes or CD4⁺ T cells were incubated for 48 hr with collagen (50 μg/ml). After the incubation, cells were collected by centrifugation and added as responders (1×10⁶/well, 2×10⁶/well, 1×10⁷/well and 2×10⁷/well). Collagen-pulsed smDCs (2×10⁵/well and 2×10⁶/well) or mDCs (2×10⁵/well and 2×10⁶/well) were seeded into 6-well plates as stimulators. The MLR was assessed to be the ratio of 1 stimulator to 5 or 10 responders (2×10⁵ stimulators: 1×10⁶ or 2×10⁶ responders, 2×10⁶ stimulators: 1×10⁷ or 2×10⁷ responders). The mixed cells were co-cultured at 37° C. for 72 hr in 2 ml of RPMI 1640 supplemented with 10% FBS. Finally, cells were harvested for measurement of T_{reg} cell population and the culture supernatants were collected for cytokine ELISA. On the other hand, in order to investigate the change of the T_{reg} cell population and the extent of DC maturation (by using CD80 and CD86 markers) according to the change of DC numbers in response to CD4⁺ T cells, 2×10⁵-2×10⁶ smDCs were co-cultured with 1×10⁷ CD4⁺ T cells for 72 hr.

### FACS Analysis

For phenotypic analysis, direct immunofluorescence was used for cell surface staining of smDCs, which were stained in FACS buffer (0.2 BSA, 0.02 sodium azide in PBS) with 1×10⁵ cells per staining. Antibody incubation was performed at 4° C. for 20 min. Data was reported as a histogram or dot plot by using FACSCalibur (BD Bioscience, Mountain View, Calif., USA) with CellQuest software. Cells were gated according to their forward and side-scattering pattern. For each marker, 10⁴ cells were counted in the gate. Additionally, FITC-conjugated Foxp3 and PE-conjugated CD25 markers were used for investigation of the T_{reg} cell population.

### Evaluation of Th1/Th2 Response

Quantitative analysis of Th1 cytokine IFN-γ and Th2 cytokine IL-4/IL-10 levels was performed by ELISA on supernatants from 72 hr-MLR cultures using 2×10⁵ DCs and CD4⁺ T cells. Additionally, quantitative analysis of TGF-β level was performed by ELISA on samples above. The commercially available ELISA kits (R&D system, Abington, OX, UK) were used as indicated by manufacturer.

### Induction of Arthritis by Using Collagen in DBA/1 Mice

Arthritis was induced as previously reported [Tetsuya Tomita, Yoshimi Kakiuchi and Philip S Tsao: THR0921, a novel peroxisome proliferator-activated receptor gamma agonist, reduces the severity of collagen-induced arthritis. Arthritis Red Ther, 2006, 8:R7.]. Briefly, 6-week old female DBA/1 mice were immunized at the base of the tail with 200 μg of chicken type II collagen dissolved in 100 μl of 0.05 M acetic acid and mixed with an equal volume (100 μl) of CFA. Collagen (2 mg/ml) was dissolved by stirring overnight at 4° C. On day 21, mice were boosted with a subcutaneous injection of CII in IFA. The booster injection was necessary to induce reproducible CIA, which normally developed at about day 34.

### In Vivo Experimental Protocol

Mice were divided into 4 groups; a group composed of CIA mice vaccinated with 2×10⁵ smDCs, a group composed of CIA mice vaccinated with 2×10⁶ smDCs, a group composed of unvaccinated CIA mice, and a control group composed of naïve mice. Starting on day 21, animals were vaccinated (injected subcutaneously in the abdominal area) with smDCs. For histological studies and evaluation of immune status, animals were sacrificed on day 41 by cervical dislocation. Additionally, for evaluation of immune status, CD4⁺ T cells isolated from the spleen of mice were cultured in the presence of 50 μg/ml collagen for 48 hr, and the T_{reg} cell population and IFN-γ/IL-4 secretion were investigated by FACS analysis and ELISA, respectively.

### Evaluation of Development of Arthritis and Histological Studies

Disease activity of the CIA was assessed visually twice a week between day 27 and 61 by two blinded observers using a three-point scale for each paw. The severity of arthritis was expressed as a mean arthritis index on a 0-3 scale (0, normal joint; 1, slight inflammation and redness; 2, severe erythema and swelling affecting the entire paw; and 3, deformed paw or joint, with ankylosis, joint rigidity, and loss of function). The total score for clinical disease activity was based on all four paws, with maximum score of 12 for each mouse [Banda N K, Kraus D, Vondracek A, Huynh L H, Bendele A, Holers V M, Arend W P: Mechanisms of effects of complement inhibition in murine collagen-induced arthritis. Arthritis Rheum, 2002, 46:3065-3075.]. Additionally, the footpad thickness was measured twice a week with a caliper. For histological studies, hind paws were removed from mice in all groups on day 41 and fixed in 10% phosphate-buffered formalin for 2 days. Thereafter, fixed samples were decalcified for 18 days in 10% formic acid, dehydrated, and embedded in paraffin blocks. Sections (5 μm) were cut along a longitudinal axis, mounted and stained with hematoxylin and eosin as previously described [Tomita T, Takeuchi E, Tomita N, Morishita R, Kaneko M, Yamamoto K, Nakase T, Seki H, Kato K, Kaneda Y, Ochi A P: Suppressed severity of collagen-induced arthritis by in vivo transfection of nuclear factor kappaB decoy oligodeoxynucleotides as a gene therapy. Arthritis Rheum, 1999, 42:2532-2542.].

### Statistical Analysis

The results are expressed as means±SD. The Mann-Whitney U test was used for all statistical analysis. A p-value of less than 0.05 was considered significant.

### Results

TNF-α-Maturated smDCs Induce in a Marked Level the T_{reg} Cell Population and Th2 Cytokine Secretion

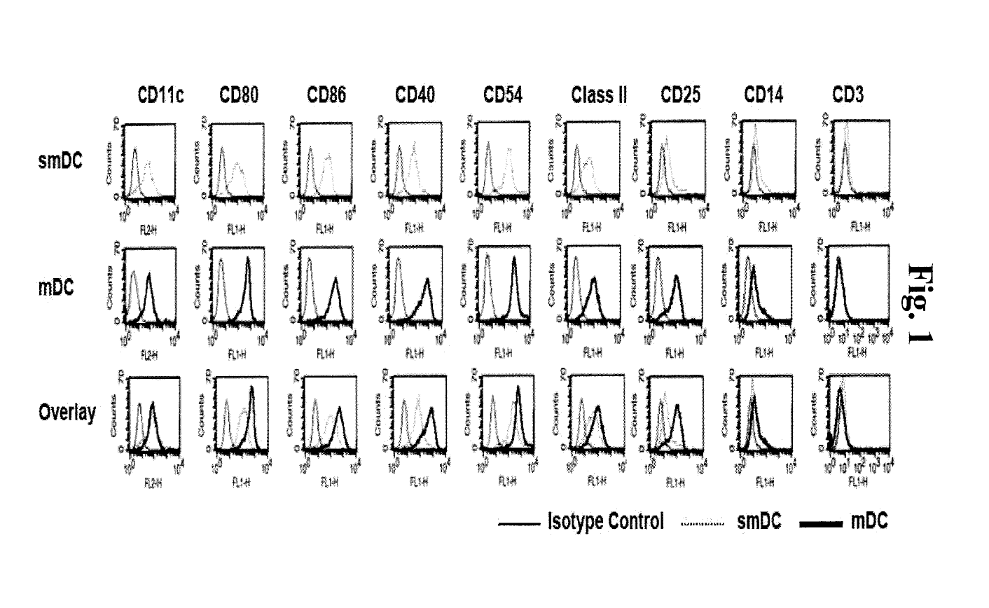

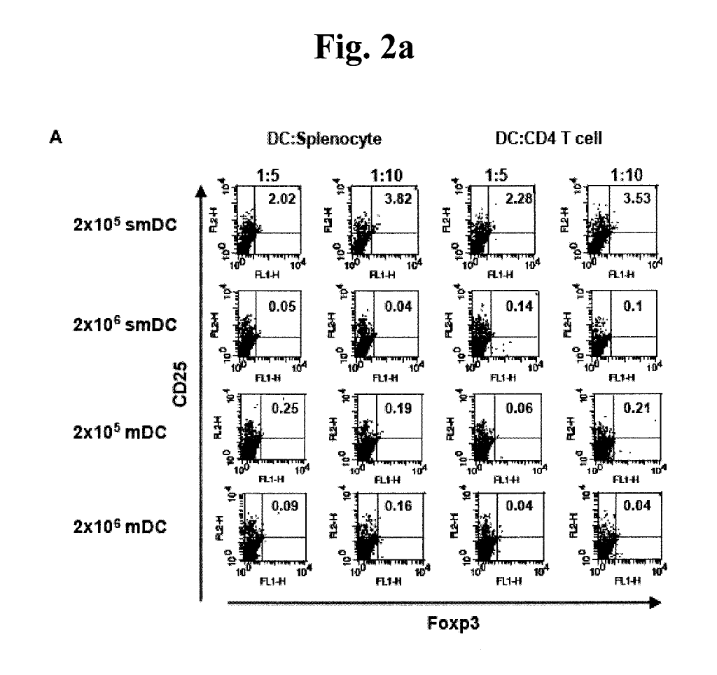

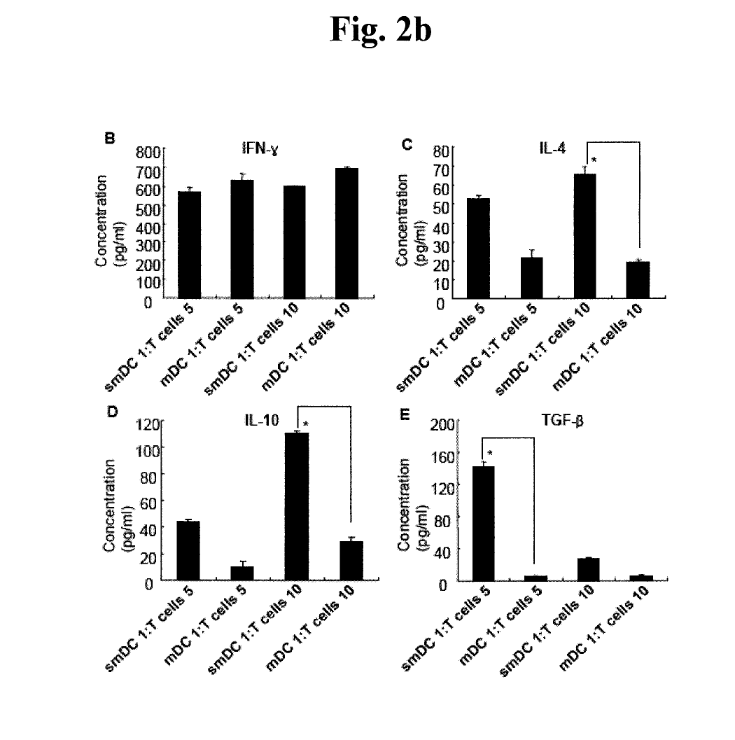

DCs include a heterogeneous family of professional antigen presenting cells (APCs) involved in initiation of immunity and in immunological tolerance. It has been found that smDCs characterized by low expression of co-stimulatory molecules, CD80, CD86, and CD40 (as compared to mDCs) (FIG. 1) markedly induced the T_{reg} cell population, Th2 cytokine IL-4/IL-10 secretion, and immune suppressive agent TGF-β in MLR with CD4⁺ T cells (FIGS. 2a and 2b). To analyze the influence of the maturational state of DCs on the priming and differentiation of naive T cells, functional assessment of smDCs and mDCs was performed by using these cells as stimulators in MLR. DCs were pulsed with collagen and subsequently treated with TNF-α for 4 h or LPS for 24 h. Using 2×10⁵ smDCs as stimulators of MLR resulted in preferential production of the Th2 cytokine IL-4 and reduction of the Th1 cytokine IFN-gamma in contrast to 2×10⁵ mDCs (FIG. 2b panel B and panel C). To assess a possible tolerogenic role of smDCs, we examined the population of T_{reg} cells and production of immunosuppressive cytokines. Using 2×10⁵ smDCs as stimulators of MLR resulted in increased T_{reg} cells (FIG. 2a) and the remarkable production of immunosuppressive cytokine IL-10 and TGF-13 (FIG. 2b panel D and panel E). These results demonstrate that smDCs have the ability to induce T_{reg} cells that produce IL-10 and TGF-β in response to collagen in vitro. On the other hand, 2×10⁶ smDCs as stimulators of MLR had no significant effect on the increase of T_{reg} cell population.

smDC Vaccination Inhibits CIA Progression in a Significant Level

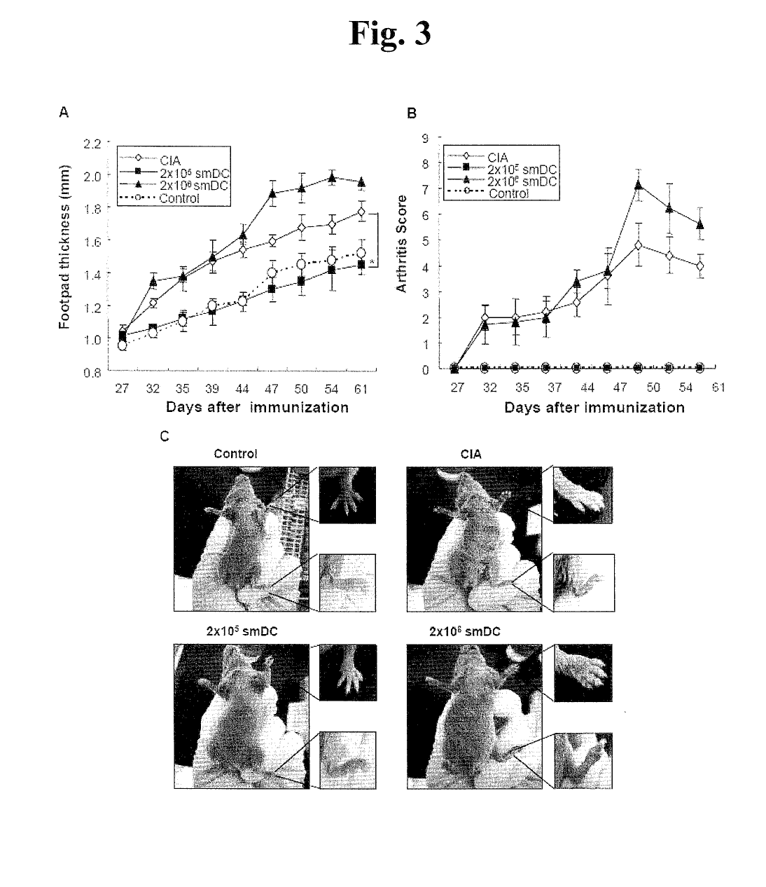

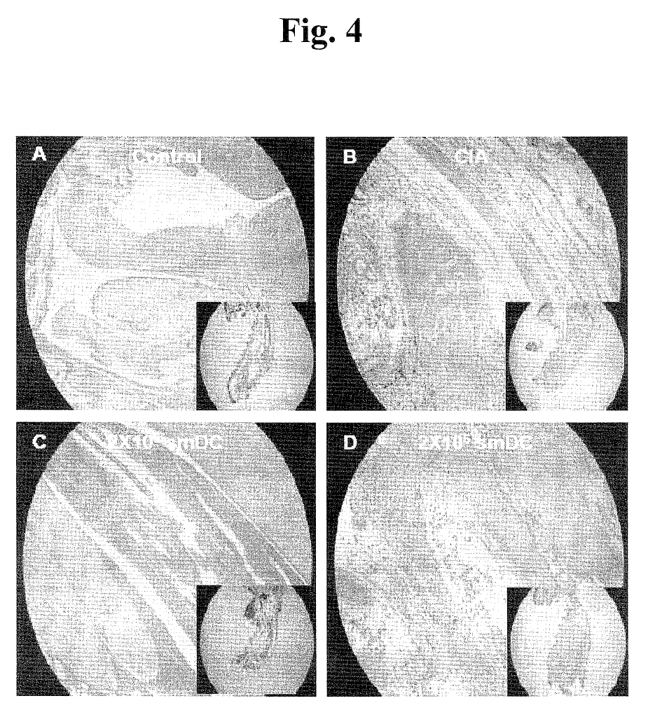

The CIA model of arthritis is a well-established method of evaluating therapeutic interventions in autoimmune arthritis. Severe induction protocols have been reported, which in essence, all induce a T cell-dependent inflammatory infiltration of the synovial membrane, leading to cartilage destruction and bone erosion. The incidence of onset of arthritis was 100% in untreated and smDC-injected CIA groups. Repeated measure analysis of variance demonstrated that smDCs abrogated the onset of arthritis during the course of experiment compared with CIA mice. We first determined a therapeutically optimal concentration of smDCs by measuring the arthritis index and foot-fad thickness by macroscopic examination of joint swelling and erythema at a 3-5 day interval. Mice were injected with 2×10⁵ smDCs or 2×10⁶ smDCs. The incidence of arthritis was not observed at all in 2×10⁵ smDC-injected animals. Consistent with this result, the arthritis index was also a zero point at that cell number (FIG. 3 panel B). When animals were injected with 2×10⁵ smDCs, the progression of arthritis was dramatically inhibited in mice injected with 2×10⁵ smDCs compared with unvaccinated control mice (FIG. 3 panel A and panel C). The increase in paw thickness was dramatically blocked in a significant level in mice injected with 2×10⁵ smDCs as compared to CIA control mice (FIG. 3 panel A). However, the progression of arthritis was further promoted in mice injected with 2×10⁶ smDCs compared to unvaccinated CIA control mice. These results were in line with in vitro MLR data (FIG. 2a panel A) and implied that administration of the appropriate number of smDCs could suppress the course of CIA in mice. Although we have demonstrated a clear inhibition of arthritis manifestation using the average arthritis score per affected paw, we further examined histological differences in vaccinated or unvaccinated CIA mice. CIA animals vaccinated or unvaccinated with smDCs (2×10⁵ or 2×10⁶) were sacrificed 41 days after arthritis onset and joints were examined in serial sections. We observed that 2×10⁶ smDCs-treated mice (FIG. 4 panel D) and CIA control mice (FIG. 4 panel B) exhibited marked inflammatory cell infiltration, pannus formation, and bone erosion. In contrast, 2×10⁵ smDC-treated mice had no any RA symptoms (FIG. 4 panel C). These results were consistent with morphologic data as above.

smDCs Potently Induce the T_{reg} Cell Population Both In Vivo and In Vitro

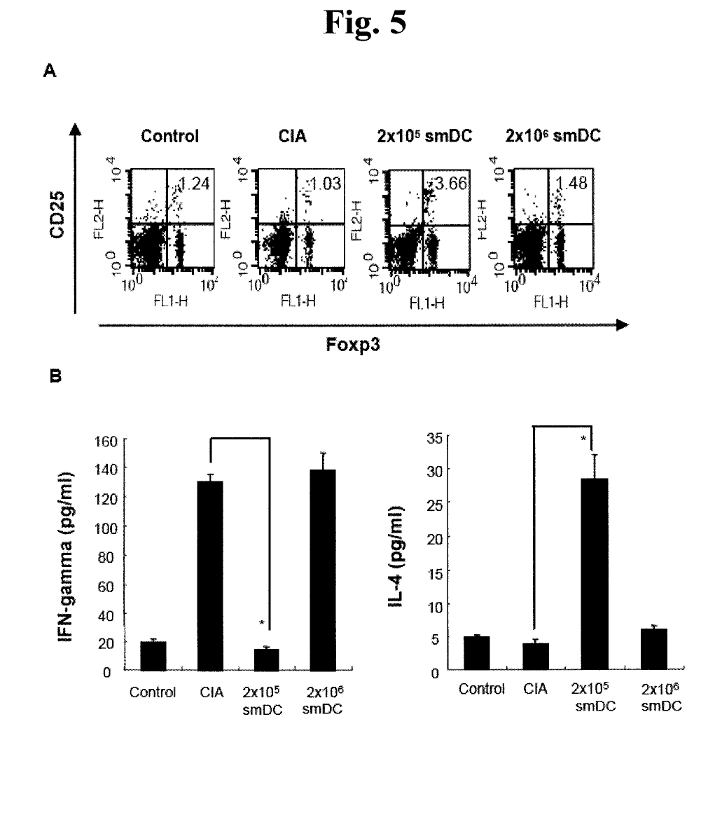

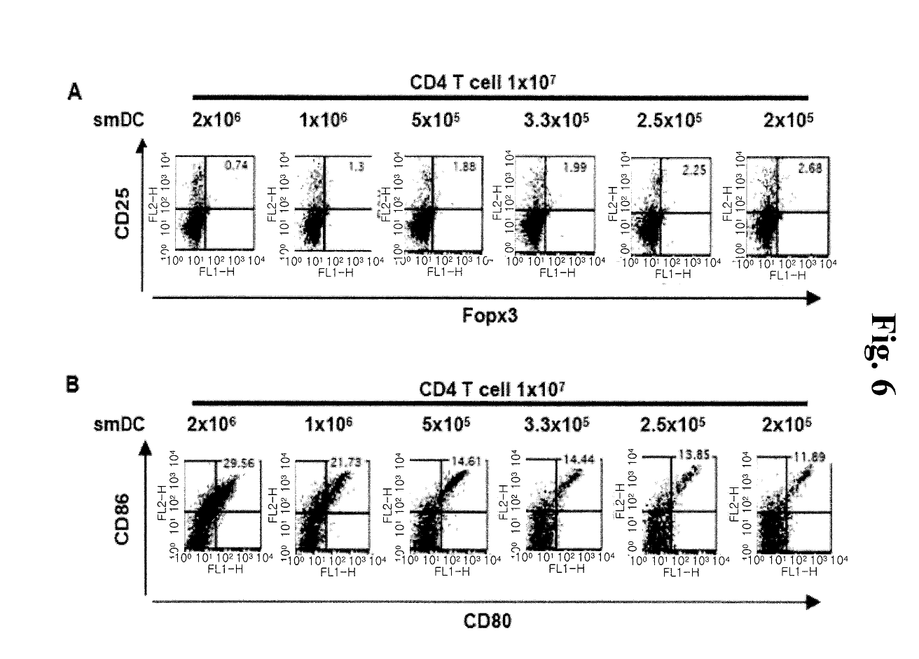

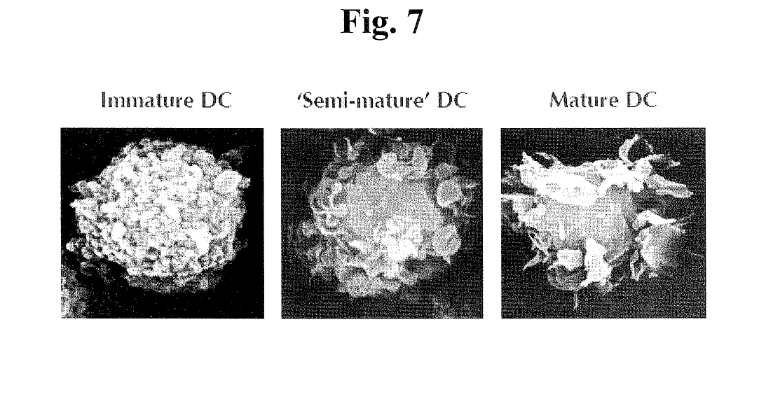

Next, we determined whether T_{reg} cells from mice vaccinated with 2×10⁵ smDCs were induced from mice vaccinated with 2×10⁵ smDCs. As shown in FIG. 5 panel A, CD4⁺CD25⁺Foxp3⁺ T_{reg} cells were remarkably induced from the spleen of mice vaccinated with 2×10⁵ smDCs. Additionally, Th1 cytokine IFN-γ and Th2 cytokine IL-4 secretions dramatically decreased and increased in a significant level, respectively, in 2×10⁵ smDC-vaccinated mice (FIG. 5 panel B). These results further support data as above (FIG. 3 and FIG. 4). Moreover, in order to elucidate the cause of contrary function dependent on smDC numbers in response to CD4⁺ T cells, the MLR with varied numbers of smDCs (2×10⁵-2×10⁶ cells) was performed. As shown in FIG. 6, the increase of Foxp3⁺T_{reg} cell population (FIG. 6 panel A) and the low expression of co-stimulatory molecules (CD80 and CD86) (FIG. 6 panel B) were dependent on the gradual decrease of smDC numbers. These results showed that the determination of DC numbers is highly important for experimental and clinical trials, and therefore careful DC application can be required for the treatment of autoimmune disease.

As described in the above, the present invention provides semi-mature dendritic cells having a remarkable activity to induce tolerogenic immune responses and a pharmaceutical composition for treating rheumatoid arthritis, which comprises the semi-mature dendritic cells as an active ingredient. The pharmaceutical composition has an effectiveness to treat or prevent autoimmune disease, in particular rheumatoid arthritis by suppressing autoimmune responses.

Having described a preferred embodiment of the present invention, it is to be understood that variants and modifications thereof falling within the spirit of the invention may become apparent to those skilled in the art, and the scope of this invention is to be determined by appended claims and their equivalents.

### REFERENCES

Tetsuya Tomita, Yoshimi Kakiuchi and Philip S Tsao: THR0921, a novel peroxisome proliferator-activated receptor gamma agonist, reduces the severity of collagen-induced arthritis. Arthritis Red Ther, 2006, 8:R7.
Banda N K, Kraus D, Vondracek A, Huynh L H, Bendele A, Holers V M, Arend W P: Mechanisms of effects of complement inhibition in murine collagen-induced arthritis. Arthritis Rheum, 2002, 46:3065-3075.
Lutz, M. B., N, Kukutsch, A. L. Ogilvie, S. Rossner, F. Koch, N. Romani, and G. Schuler: An advanced culture method for generating large quantities of highly pure dendritic cells from mouse bone marrow. J. Immunol. Methods, 1999, 223:77-92.
Jonuleit H, Schmitt E, Schuler G, Knop 3, Enk A H: Induction of interleukin 10-producing, nonproliferating CD4(+) T cells with regulatory properties by repetitive stimulation with allogeneic immature human dendritic cells. J Exp Med, 2000, 192: 1213-1222.
Tomita T, Takeuchi E, Tomita N, Morishita R, Kaneko M, Yamamoto K, Nakase T, Seki H, Kato K, Kaneda Y, Ochi A P: Suppressed severity of collagen-induced arthritis by in vivo transfection of nuclear factor kappaB decoy oligodeoxynucleotides as a gene therapy. Arthritis Rheum, 1999, 42:2532-2542.
Menges, M., S. Rossner, C. Voigtlander, H. Schindler, N. A. Kukutsch, C. Bogdan, K. Erb, G. Schuler, and M. B. Lutz: Repetitive injections of dendritic cells matured with tumor necrosis factor-α induce antigen-specific protection of mice from autoimmunity. J. Exp. Med, 2002, 195: 15-21.
Verqinis P, Li H S, Caravanniotis G: Tolerogenic semimature dendritic cells suppress experimental autoimmune thyroiditis by activation of thyroglobulin-specific CD4⁺CD25⁺ T Cells. J Immunol, 2005, 174:7433-7442.

1. A semi-mature dendritic cell for treating rheumatoid arthritis.

2. The semi-mature dendritic cell according to claim 1, wherein the semi-mature dendritic cell has reduced expression level of one or more of co-stimulating factors of CD80, CD86 and CD40 compared to a mature dendritic cell.

3. A pharmaceutical composition for treating rheumatoid arthritis, which comprises (a) a pharmaceutically effective amount of a semi-mature dendritic cell; and (b) a pharmaceutically acceptable carrier.

4. The pharmaceutical composition according to claim 3, wherein the semi-mature dendritic cell is obtained by culturing immature dendritic cells in the presence of TNF (tumor necrosis factor)-α and specific antigens of rheumatoid arthritis.

5. The pharmaceutical composition according to claim 3, wherein the semi-mature dendritic cell has reduced expression level of one or more of co-stimulating factors of CD80, CD86 and CD40 compared to a mature dendritic cell.

6. The pharmaceutical composition according to claim 3, wherein the semi-mature dendritic cell has a potential to increase the population of a CD4⁺CD25⁺Foxp3⁺ regulatory T cell.

7. The pharmaceutical composition according to claim 3, wherein the semi-mature dendritic cell has a potential to suppress the secretion of Th1 cytokine IFN-γ and to promote the secretion of Th2 cytokine IL-4 or IL-10.

8. The pharmaceutical composition according to claim 3, wherein the semi-mature dendritic cell has a potential to promote the secretion of immunosuppressive cytokine TGF-β.

9. A method of treating rheumatoid arthritis in a subject, the method comprising administering a semi-mature dendritic cell to the subject.

10. The method according to claim 9, wherein the semi-mature dendritic cell is obtained by culturing immature dendritic cells in the presence of TNF (tumor necrosis factor)-α and specific antigens of rheumatoid arthritis.

11. The method according to claim 9, wherein the semi-mature dendritic cell has reduced expression level of one or more of co-stimulating factors of CD80, CD86 and CD40 compared to a mature dendritic cell.

12. The method according to claim 9, wherein the semi-mature dendritic cell has a potential to increase the population of a CD4⁺CD25⁺Foxp3⁺ regulatory T cell.

13. The method according to claim 9, wherein the semi-mature dendritic cell has a potential to suppress the secretion of Th1 cytokine IFN-γ and to promote the secretion of Th2 cytokine IL-4 or IL-10.

14. The method according to claim 9, wherein the semi-mature dendritic cell has a potential to promote the secretion of immunosuppressive cytokine TGF-β.
